(19) United States
(12) Patent Application Publication
(10) Pub. No.: US 2004/0086895 A1
(43) Pub. Date: May 6, 2004

(54) Method of electrochemical detection of somatic cell mutations

(76) Inventors: Donald M. Crothers, Northford, CT (US); R. Erik Holmlin, San Diego, CA (US); Chunnian Shi, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/429,293

(22) Filed: May 2, 2003

Related U.S. Application Data
(63) Continuation-in-part of application No. 10/424,542, filed on Apr. 24, 2003.
(60) Provisional application No. 60/424,656, filed on Nov. 6, 2002.

(51) Int. Cl. C12Q 1/68; C12P 19/34

(52) U.S. Cl. 435/6; 435/91.2

(57) ABSTRACT

The present disclosure relates to the detection of somatic cell mutations, particularly as part of a method to screen for cancer or precancer. The disclosure includes techniques for extracting and isolating oligonucleotides from a patient and conducting hybridization assays. Preferred embodiments include a combination of the following steps: extracting a biological sample from a patient, purifying a nucleic acid from a biological sample, amplifying a nucleic acid, isolating a nucleic acid in single stranded form, cyclizing a nucleic acid, elongating a nucleic acid, controlling hybridization stringency, amplifying a nucleic acid on a chip, and detecting hybridization.

### RELATED APPLICATIONS

This application claims priority from U.S. Pat. Application No. 60/424,656 entitled UNIVERSAL TAG ASSAY filed Nov. 6, 2002. This application also claims priority from, and is a continuation-in-part application of U.S. patent application Ser. No. ______ entitled “UNIVERSAL TAG ASSAY,” filed Apr. 24, 2003, attorney docket number GENOM.019A. The subject matter of the aforementioned applications is hereby incorporated by reference in its entirety.

### BACKGROUND OF THE INVENTION

1. Field of the Invention

The present invention relates to the detection of genetic mutations in somatic cells and methods of screening patients for cancer or precancer.

2. Description of the Related Art

Somatic cells are definitionally distinguished from germ cells as the former are the cells which make up an individual's body while the latter are those cells which can participate in sexual reproduction. Both types of cells can experience genetic mutations under a variety of circumstances. Mutations in somatic cells are typically not passed to an individual's offspring; they are, however, often passed within an individual to the daughter cells of the mutated somatic cell through mitosis. The frequency with which somatic cells reproduce is generally related to the type of cell and to various abnormalities caused by genetic mutations in the cell. The propagation of somatic cell mutations is a principal mechanism behind most cancers.

If a mutation in a somatic cell increases the rate of its reproduction in an uncontrolled manner, then the number of daughter cells may increase rapidly in that area. When this occurs, the daughter cells often divide before reaching their mature state. This can result in an ever increasing number of cells that have no beneficial function to the body, yet absorb body nutrition at an increasing rate. Tissue of this type may be referred to as a tumor. If the cells remain in their place of origin and do not directly invade surrounding tissues, the tumor is said to be “benign.” If the tumor invades neighboring tissue and causes distant secondary growths (called metastasis), it may be termed “malignant.”

The adverse health consequences of a tumor depend on the tissue in which it is located, how rapidly it grows, and how quickly it is detected and treated. Numerous environmental factors can contribute to somatic cell mutations, though mutations can arise spontaneously as well. Mutations owing to either origin may ultimately produce tumors.

Various techniques for detecting mutations are known in the art. For example, techniques for detecting colorectal cancer are disclosed in U.S. Pat. No. 5,741,650 (Lapidus, et al.); U.S. Pat. No. 5,834,181 (Shuber); U.S. Pat. No. 5,849,483 (Shuber); U.S. Pat. No. 5,952,178 (Lapidus et al.); U.S. Pat. No. 6,268,136 (Shuber et al.); U.S. Pat. No. 6,303,304 (Shuber et al.); U.S. Pat. No. 6,428,964 (Shuber); all of which are hereby incorporated by reference in their entirety.

Present techniques for studying somatic cell mutations typically allow identification of an affected genetic region to within 10 to 1000 base pairs, while the precise position and nature of the nucleotide change remain elusive. Presently, there is no reliable, inexpensive method for rapidly locating and characterizing genetic mutations occurring in somatic cells.

Such a method could facilitate earlier and more effective treatments for patients having, or at risk of developing, mutation-related disorders, including various cancers. Accordingly, what is needed in the art is a method that is capable of identifying the location and nature of nucleotide changes that occur within somatic cells.

### SUMMARY OF THE INVENTION

One aspect of the invention is a method for detecting a target polynucleotide, including the steps of: synthetically producing an enlarged target polynucleotide; hybridizing the target polynucleotide to a probe polynucleotide in a detection zone; and detecting the amount of polynucleotide in the detection zone to ascertain whether target polynucleotide has hybridized in the detection zone. In some cases, target polynucleotide is enlarged by attaching one or more polynucleotide strands to the target. Further the target polynucleotide can be enlarged by attachment of a plurality of polynucleotide strands, producing a branched structure. In some embodiments, the target polynucleotide is hybridized to more than one probe polynucleotide in the detection zone.

A further aspect of the invention is a method for detecting a nucleic acid analyte, including: generating an elongated reporter nucleic acid if the nucleic acid analyte is present; capturing the reporter nucleic acid with an immobilized probe that is substantially shorter than the reporter nucleic acid; and generating a signal that is a function of the size of the captured reporter nucleic acid to indicate the presence or absence of the nucleic acid analyte.

### BRIEF DESCRIPTION OF THE DRAWINGS

FIG. 1A depicts short strand duplex melting curves.

FIG. 1B depicts long strand duplex melting curves.

FIG. 1C depicts melting curves in which an elongated target strand is hybridized to multiple short strand probes.

FIG. 2A illustrates on-chip amplification using head-to-tail polymerization.

FIG. 2B illustrates on-chip amplification using rolling circle amplification.

FIG. 2C illustrates on-chip amplification using a branch technique in conjunction with rolling circle amplification.

### DETAILED DESCRIPTION OF THE PREFERRED EMBODIMENTS

The present invention is generally related to the detection of somatic cell mutations. Preferred embodiments include the isolation of oligonucleotides from biological samples and an analysis of various oligonucleotide sequences for complementarity using an electrochemical hybridization assay. Accordingly, the knowledge that an oligonucleotide of unknown sequence is complementary to an oligonucleotide of known sequence can be used to identify the unknown sequence. Similarly, comparing to an oligonucleotide of unknown sequence to an oligonucleotide known to be healthy or “wild” can be used to characterize the unknown sequence as either wild or mutated.

Various techniques for isolating oligonucleotides and conducting hybridization assays are described in copending U.S. Pat. Application Serial No. 60/424,656, filed Nov. 6, 2002; U.S. patent application Ser. No. ______ entitled “UNIVERSAL TAG ASSAY,” filed Apr. 24, 2003, attorney docket number GENOM.019A; U.S. patent application Ser. No. ______ entitled “ELECTROCHEMICAL METHOD TO MEASURE DNA ATTACHMENT TO AN ELECTRODE SURFACE IN THE PRESENCE OF MOLECULAR OXYGEN,” filed simultaneously herewith, attorney docket number GENOM.023A; all of which are hereby incorporated by reference in their entirety.

Preferred embodiments of the present invention include the detection of polynucleotide hybridization in a detection zone. Particularly preferred embodiments feature the use of a ruthenium complex in conducting an electrochemical assay. Preferably, such an electrochemical assay detects nucleic acid hybridization using the general technique of Steele et al. (1998, Anal. Chem. 70:4670-4677), hereby expressly incorporated by reference in its entirety.

Typically, in carrying out this technique, a plurality of nucleic acid probes which are complementary to a sequence of interest are used. In certain preferred embodiments, probes range in length from about 10 to 25 base pairs, with a length of about 17 base pairs being most preferred. Preferably, the probe strands are positioned within a detection zone. In particularly preferred embodiments, the detection zone includes a surface, such as an electrode, in contact with a liquid medium, wherein the probe strands are immobilized on the surface such they are also in contact with the liquid medium. Preferably, the surface is a gold or carbon electrode that is coated with a protein layer such as avidin or streptavidin to facilitate the attachment of the nucleic acid probe strands to the electrode. This protein layer should be porous, such that it allows ions to pass from the liquid medium to the electrode and vice versa. When attaching a probe strand to an avidin layer, it is preferable to first bind the probe strand covalently to a biotin complex and then allow the biotin to attach to the avidin. Alternatively, probe strands can be attached directly to the surface, for example by using a thiol linkage to covalently bind nucleic acid to a gold electrode. Carbon electrodes or electrodes of any other suitable conductor can also be used.

In further carrying out this technique, a target strand (a nucleic acid sample to be interrogated relative to the probe) can be contacted with the probe in any suitable manner known to those skilled in the art. For example, a plurality of target strands can be introduced to the liquid medium described above and allowed to intermingle with the immobilized probes. Preferably, the number of target strands exceeds the number of probe strands in order to maximize the opportunity of each probe strand to interact with target strands and participate in hybridization. If a target strand is complementary to a probe strand, hybridization can take place. Techniques for adjusting the stringency of hybridization and techniques for detecting hybridization are also discussed herein.

Further, embodiments of the present invention can include any combination of the following steps: extracting a biological sample from a patient, purifying a nucleic acid from a biological sample, amplifying a nucleic acid, isolating a nucleic acid in single stranded form, cyclizing a nucleic acid, elongating a nucleic acid, controlling hybridization stringency, amplifying the nucleic acid on a chip, and detecting hybridization. Accordingly, preferred embodiments for each of these steps are discussed in the following sections.

In the present disclosure, references to extracting an oligonucleotide from a patient typically refer to obtaining a sequence that will form the basis of a target strand. However, in many embodiments, the same techniques, or those which are similar, will also be appropriate for obtaining a sequence that will form the basis of a probe strand. Those of skill in the art will recognize that various biological and/or artificial sources of oligonucleotides are available and will be able to decide which are most suitable for creating probes or targets depending on the particular goals of the assay to be conducted.

Extracting a Biological Sample

In accordance with the present invention, a variety of methods for extracting nucleic acid from various biological samples from a patient can be used. Biological samples that are useful in the present invention can include any sample from a patient in which a nucleic acid is present. Such samples can be prepared from a any tissue, cell, or body fluid. Examples of biological cell sources include blood cells, colon cells, buccal cells, cervicovaginal cells, epithelial cells from urine, fetal cells or cells present in tissue obtained by biopsy. Exemplary tissues or body fluids include sputum, pancreatic fluid, bile, lymph, plasma, urine, cerebrospinal fluid, seminal fluid, saliva, breast nipple aspirate, pus, amniotic fluid and stool. Useful biological samples can also include isolated nucleic acid from a patient. Nucleic acid can be isolated from any tissue, cell, or body fluid using any of numerous methods that are standard in the art.

In some preferred embodiments, a stool sample is taken from a patient as part of a method of screening for colorectal cancer. In particular, methods of extracting biological samples from stool are described in U.S. Pat. No. 5,741,650 (Lapidus et al.), herein incorporated by reference. Lapidus et al. teach sectioning a stool sample to extract cells and cellular debris that may be indicative of cancer or precancer. Such a method can be used to obtain biological material containing a nucleic acid for further use in accordance with the present invention.

Purifying Nucleic Acid from a Sample

Once a sample has been extracted from a patient, a variety of techniques can be used to purify nucleic acid. Suitable nucleic acids can include DNA and RNA. The particular nucleic acid purification method will typically depend on the source of the patient sample. Techniques for purifying nucleic acid are known in the art and can include the use of homogenization, centrifugation, extraction with various solvents, chromatography, electrophoresis, and other known techniques.

In some preferred embodiments, the biological sample is a stool sample and nucleic acid from colorectal tissue is isolated and purified from stool cross sections according to methods disclosed in U.S. Pat. No. 6,406,857 (Shuber et al.), hereby expressly incorporated by reference in its entirety.

Amplification of Nucleic Acid

Various techniques which are known in the art can be used to amplify a nucleic acid when practicing the present invention. RCA is one technique that can be used, through PCR is preferred. It is particularly advantageous to use a “digital PCR” technique. Digital PCR refers to a PCR method in which a liquid sample containing nucleic acids of interest is so thoroughly diluted and partitioned that each partition contains at most one nucleic acid molecule. Accordingly, if subsequent PCR amplification on a partition is successful, all of the resulting strands will be derived from one strand. Hence all of the PCR products for a given partition will be identical. Because the partitions themselves are unlikely to be identical to all the other partitions, it will often be advantageous to study those partitions found to contain nucleic acids in separate assays to determine which warrant further attention.

Digital PCR is discussed in greater detail in Vogelstein et al. “Digital PCR,” Proc. Natl. Acad. Sci. USA, Vol. 96, pp. 9236-41, August 1999; which is hereby incorporated by reference in its entirety.

Isolating Single Stranded Nucleic Acid

Various techniques are known in the art for producing or isolating single stranded nucleic acid from samples containing double stranded nucleic acid.

In one preferred method, single stranded nucleic acid is isolated using a streptavidin-coated bead. In performing this technique, an amplification product is denatured to generate single-stranded products, wherein at least one strand contains an addressable ligand at one terminus. In some preferred embodiments, a biotinylated single-stranded PCR product having a copy of the nucleotide sequence of interest is incubated with streptavidin-coated beads, under conditions such that the biotinylated PCR product is attached to a bead, forming a bead-target sequence complex.

In other preferred embodiments, one strand of a double stranded nucleic acid is removed, for example, by selective exonuclease digestion. The remaining single stranded nucleic acid can further be used in accordance with the present invention.

Cyclizing the Nucleic Acid and Performing RCA

In performing an assay in accordance with the present invention, it is possible to cyclize and elongate the target nucleic acid prior to hybridization. “Cyclization” generally refers to the process of creating a polynucleotide circle (preferably containing a particular sequence), while “elongation” generally refers to the process of increasing the length of a polynucleotide. In preferred embodiments, elongation includes a rolling circle amplification (RCA) step with an appropriate polynucleotide circle and is used to create a long strand of target nucleic acid.

In particular, cyclization and elongation can be used to generate one or more long target strands in which a sequence being interrogated is repeated several times. Effectively, many copies of a small target strand are linked end to end to generate a large target strand. Although cyclization/elongation can be used to add as little as one repetition, it is generally preferred that multiple repetitions be added, for example, approximately 10, 50, 100, 250, 500, 750, or 1000 repetitions or more may be attached. Circle size is also adjustable according to the requirements of the assay. Preferred circle sizes are in the range of about 40 to about 1000 base pairs, with about 800 base pairs being most preferred. Notably, the number of repetitions selected can depend on the length of the circle being used. Specifically, it will generally be preferable to use more repetitions with smaller circles and fewer repetitions with larger circles so that the strands produced will be appropriately manageable and functional according to the demands of the assay.

Generally, any one of the many repetitions of the sequence on a large strand would be able to hybridize to a probe just as if that sequence were alone on a standard short target strand. Further, just one large target strand can generally hybridize to multiple probes (by coiling back toward the electrode surface and allowing another identical region of the long strand to attach to another complementary probe).

Elongation and the use of long target strands has various advantages. Particularly favorable advantages are related to stringency. “Stringency” refers to a measurement of the ease with which various hybridization events can occur. For example, two strands that are perfectly complementary generally form a more stable hybrid than two strands that are not. Various stringency factors (such as temperature, pH, or the presence of a species able to denature various hybridized strands) can be adjusted such that in a single environment, the perfectly complementary pair would stay together while the imperfect pair would fall apart. Ideal conditions are generally those which strike a balance between minimizing the number of hybridizations between noncomplementary strands (false positives) and minimizing the number of probes which remain unhybridized despite the presence of eligible complementary target strands (false negatives). Other various techniques for controlling stringency are also discussed in the next section.

Elongation is one technique that is useful in improving the effectiveness of temperature as a stringency factor. A perfect hybrid is typically more stable than an imperfect hybrid and will outlast the imperfect hybrid when the temperature is increased. However, dehybridization in either case is not a single event when dealing with populations of molecules. Instead, more and more molecules give up the hydrogen bonds that hold opposing base pairs together over a range of temperature. Perfect hybrids outlast imperfect hybrids, but it is often very difficult, if not impossible, to find a single temperature at which there are no imperfect hybrids while perfect hybrids abound.

It has been discovered that longer nucleic acid molecules exhibit a less gradual transition between their hybridized and unhybridized states when the temperature is changed. This is to say that the melt curve for a given population of molecules is steeper and more decisive when the nucleic acid strand is longer. However, the distance between the curves of perfect and imperfect hybrids of equivalent length tend to crowd in a smaller temperature range, frustrating the initial attempt to create a stringency environment that will distinguish between them.

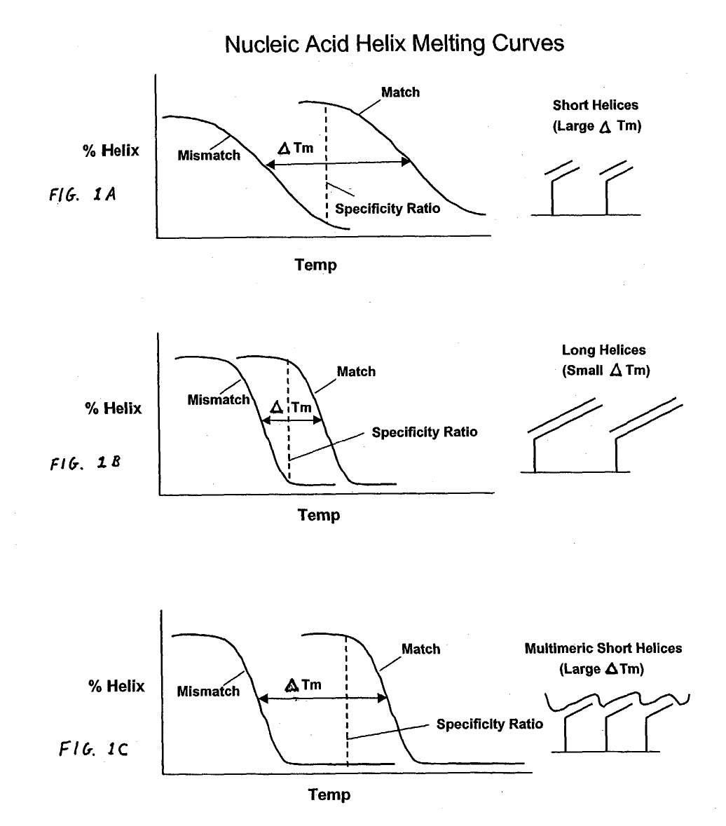

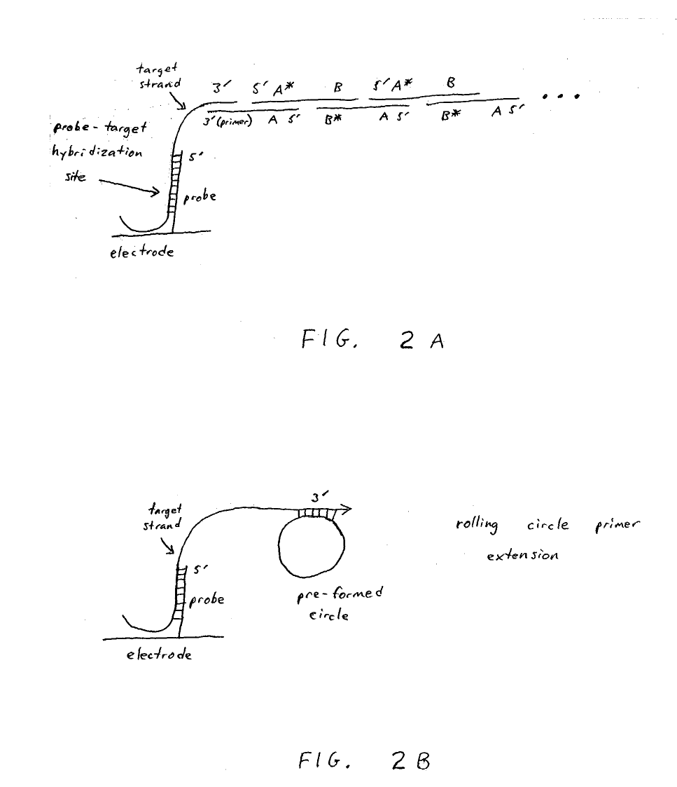

FIGS. 1A and 1B illustrate this phenomenon. FIG. 1A shows the melt curves of matched and mismatched short strand duplexes. FIG. 1B shows the melt curves for longer strands. FIG. 1A has a large ΔTm, but the gradual melt of the duplexes makes it difficult to select and maintain a temperature range that has a maximum specificity ratio. FIG. 1B has steeper and more decisive melting curves, but the ΔTm is very small, again making it difficult to select and maintain a temperature range that allows maximum specificity.

It has further been discovered that the use of elongated target strands which can hybridize to multiple probes enable a larger stringency range. In other words, the melt curves are steeper (than those of the short molecules) and that the distance between the melting temperatures of perfect and imperfect strands are farther apart (than those of the long molecules). This type of hybridization is depicted in FIG. 1C. As shown, the melt curves are steep and the ΔTm is large. This combination facilitates improved specificity in the assay because of the large temperature range in which matched duplexes generally exist and mismatched duplexes do not.

Accordingly, some embodiments of the present invention include cyclization and elongation steps to produce a target strand of increased length. Preferably, a sequence is repeated several times on the target strand such that one target strand can participate in hybridization with more than one immobilized probe. As this can be used to create a larger temperature window in which perfect hybrids remain and imperfect hybrids fall apart, it is advantageous to adjust the temperature of the assay environment to minimize false positives as well as false negatives.

In some embodiments, it is advantageous to perform an assay in which hybridization is evaluated at two or more different temperatures. For example, where a duplex polynucleotide with a single base mismatch has a melting temperature Tₘ₁ and a duplex polynucleotide with no base mismatch has a higher melting temperature Tₘ₂, it is possible to first detect whether the duplex exists at a temperature below Tₘ₁, then increase the temperature of the assay environment above Tₘ₁ to detect whether the duplex exists at a temperature between Tₘ₁ and Tₘ₂. In this case, the results of such an assay could indicate whether a single base mismatch exists in the duplex being interrogated. In some cases, a determination of the temperature at which a duplex falls apart can be used to evaluate the quantity, type, and/or location of mismatches, if any. Various techniques for detecting hybridization are discussed infra.

Those of skill in the art will appreciate that other techniques to elongate nucleic acids, including for example, head-to-tail polymerization, can also be used to achieve favorable results with regard to temperature stringency.

In some embodiments, it is advantageous to use “padlock probe” and/or “addressed amplicon” techniques when generating a target strand that can be hybridized to a probe strand. These techniques are discussed in greater detail in U.S. patent application Ser. No. ______ entitled “UNIVERSAL TAG ASSAY,” filed Apr. 24, 2003, attorney docket number GENOM.019A; herein expressly incorporated by reference in its entirety. Some embodiments of the present invention include providing a polynucleotide sample and then performing an assay to determine whether it contains a sequence of interest. In some such assays, a nucleic acid circle is prepared in connection with the polynucleotide sample that contains both a portion of a sequence complementary to the sequence being interrogated and an “address sequence.” The address sequence is typically an arbitrary sequence of nucleotides that will also appear on a probe strand. The circle can be amplified by RCA to produce a long target that contains several repetitions of the complement to the address sequence. When the target strand is allowed to interact with a probe containing the address sequence, the two can hybridize. Detection of hybridization can be used as an indication of the presence of the sequence being interrogated in the original sample. It will be appreciated that assays of this type can detect the presence of various sequences as well as the presence of single nucleotide polymorphisms (SNPs). When detecting SNPs, for example, it can be advantageous to use different cyclizable strands containing each of the possible nucleotides at the suspected SNP location. Each cyclizable strand should also have a unique address sequence. The one cyclizable strand that fits correctly with the sample can then cyclize and undergo amplification. Then, by determining which address sequence corresponds to a probe-target hybrid, the identity of the nucleotide at the SNP location can be determined.

Controlling Hybridization Stringency

In performing a hybridization step, it is preferable to introduce single stranded targets derived as described above to the liquid medium such that they may hybridize with probes immobilized on an electrode. Preferably, the number of target strands used in an assay will exceed the number of probe strands in order to maximize the opportunity of each probe strand to interact with target strands and participate in hybridization. If a target strand is complementary to a probe strand, hybridization can take place when the two come into contact. However, in some cases, even strands which are not truly complementary may come together and stay together as an imperfect hybrid. Whether or not various hybridization events occur can be influenced by various stringency factors such as temperature, pH, or the presence of a species able to denature various hybridized strands. Increasing the quantity of target strands is one technique that can be useful in minimizing the number of probes that should hybridize to targets, but do not (false negatives).

Preferred techniques for controlling stringency include setting and maintaining the temperature and pH of the liquid medium environment. More preferred techniques also incorporate introducing one or more chemical species as stringency agents that can minimize the number of false positives and/or false negatives. Agents that can be used for this purpose include quaternary ammonium compounds such as tetramethylammonium chloride (TMAC).

TMAC is particularly useful in minimizing false positives. This species generally acts through a non-specific salt effect to reduce hydrogen-bonding energies between G-C base pairs. At the same time, it binds specifically to A-T pairs and increases the thermal stability of these bonds. These opposing influences have the effect of reducing the difference in bonding energy between the triple-hydrogen bonded G-C based pair and the double-bonded A-T pair. One consequence is that the melting temperature of nucleic acid hybrids formed in the presence of TMAC is solely a function of the length of the hybrid. A second consequence is an increase in the slope of the melting curve for each probe. Together, these effects allow the stringency of hybridization to be increased to the point that single-base differences can be resolved, and non-specific hybridization minimized. Various techniques for using a stringency agent such as TMAC are discussed in U.S. Pat. No. 5,849,483 (Shuber), herein incorporated by reference.

Further, specific control of stringency factors can be useful in assays which seek to identify mutations occurring at the end of an oligonucleotide fragment. For example, the mutator cluster region of the APC gene, wherein mutations are highly correlated with colon cancer, is approximately 800 base pairs in length. If a probe oligomer is approximately 17 base pairs in length, it will typically require approximately 44 oligomers to blanket the entire 800 base pair strand. Mutations at the end of a fragment are often difficult to detect, so it can be beneficial to use a second series of oligonucleotides that also blanket that 800 base pair strand, but are offset such that the middle of the second series of oligonucleotides corresponds to the ends of the adjacent first series of oligonucleotides. Allowing for a gap of three base pairs between adjacent probe sequences, it will typically require 80 oligomers to test 800 base pairs for mutations. Various high volume techniques for testing a mutator cluster region can be used. In a preferred embodiment, standard multiwell plates having 96 wells and 20 electrodes per well can be used to test a particular region; assuming four wells are used to determine which one of the four bases appears at a particular point in the sequence, each 96 well plate can test the properties of 24 different molecules.

Further, temperature dependence can be adjusted by varying the length of individual oligonucleotides since longer sequences tend to be more stable. Oligonucleotides that are to be used in an assay need not all be the same length.

Amplifying the Hybridized Nucleic Acid

In practicing the present invention, it will sometimes be advantageous to augment the signal created by the target strand that indicates hybridization has occurred. One method for doing this is to elongate the target strand after it has hybridized to the probe. This technique may be referred to as “on-chip” amplification. Two methods for on-chip amplification are particularly preferred.

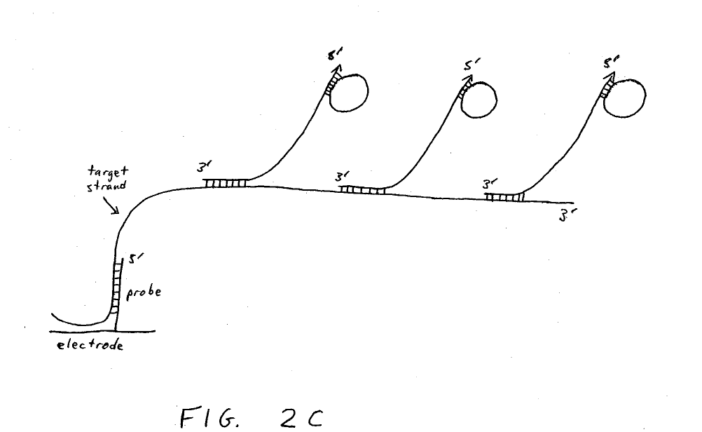

The first preferred method of on-chip amplification is depicted in FIG. 2A. Here, either the 3′ or 5′ end of the hybridized PCR product can be targeted for a head-to-tail polymerization that builds up the amount of DNA on the electrodes. Typically, three different oligonucleotides (not counting the immobilized probe and the target strands) will be used as shown here: the first oligomer is complementary to the 3′ end of the hybridized PCR product (targeting the complement of the primer sequence), and contains a sequence A at its 5′ end; the second oligomer has a sequence 5′-A*B-3′, where A* is complementary to A; the third oligonucleotide has sequence 5′-AB*-3′. As depicted in FIG. 2A, these oligomers can form a polymeric product as shown. The head-to-tail polymerization can continue until the strand reaches a desired length. Generally, when performing head-to-tail polymerization, the ultimate length of the polynucleotide is limited in part by a competing cyclization reaction of the head-to-tail oligomers. A higher concentration of head-to-tail oligomers in the liquid medium will generally produce longer linear polymers attached to the electrode, however.

The second preferred method of on-chip amplification is depicted in FIG. 2B. This method uses rolling circle amplification. Preferably, a preformed circle (approximately 40 to 300 nucleotides) that has a region complementary to the 3′ end of the bound PCR product is hybridized to the PCR product as shown. A processive DNA polymerase can then be added so that RCA results, elongating the bound PCR product. Preferably, the PCR product is elongated by approximately 10 to 100 copies of the circle.

A further technique for on-chip amplification is depicted in FIG. 2C. This technique may be used in conjunction with other on-chip amplification methods and is commonly referred to as “branch” amplification. Here, additional polynucleotides that are capable of hybridizing with the target strand in a region beyond the probe-target hybridization region can be added to the liquid medium and allowed to hybridize with the bound target to further increase the amount of bound polynucleotide material when probe-target hybridization occurs. Preferably, these branch polynucleotide strands are further amplified, for example by RCA as depicted in FIG. 2C. Further, when a branch amplification technique is used, it can be advantageous to attach branches on top of branches, a technique known as hyperbranching. Additional discussion of branching and hyperbranching techniques can be found, for example, in: Urdea, Biotechnology 12:926 (1994); Horn et al., Nucleic Acids Res. 25(23):4835-4841 (1997); Lizardi et al., Nature Genetics 19, 225-232 (1998); Kingsmore et al. (U.S. Pat. No. 6,291,187); Lizardi et al. (PCT application WO 97/19193); all of which are hereby incorporated by reference.

After performing an on-chip amplification, the increased amount of DNA can generate a larger and more detectable signal. This can be advantageous for assay purposes since both the probe and the target typically produce some detectable signal. If the signal of the target is enhanced, the contrast between hybridized and unhybridized probes will be more profound. In some embodiments, however, nucleic acid analogs can be used as probes which do not contribute to the overall signal; such designs are discussed in the following section. Even when such nucleic acid analogs are used as probes, target elongation can still be desirable.

Preferably, nucleic acid hybridization is tested electrochemically using a transition metal complex. More preferably, hybridization is detected by measuring the reduction of a ruthenium complex as described below.

Detecting Hybridization

Various techniques can be used to determine whether hybridization has occurred. As indicated above, preferred embodiments of the present invention feature the use of a transition metal complex. In particular, a ruthenium complex can be used as a counterion to conduct an electrochemical assay using the general technique of Steele et al. (1998, Anal. Chem. 70:4670-4677), herein incorporated by reference.

Counterions, such as Ru(NH₃)₆³⁺ or Ru(NH₃)₅py³⁺, can be introduced to the liquid medium surrounding the immobilized oligonucleotides. Typically, Ru(NH₃)₅py³⁺ is preferred because its reduction to a divalent ion does not occur at the same electrical potential as the reduction of molecular oxygen.

Once introduced, the counterions will tend to cloud around the negatively charged backbones of the various nucleic acid strands. Generally, the counterions will accumulate electrostatically around the phosphate groups of the nucleic acids whether they are single or double stranded. However, because a probe and target together physically constitute a larger amount of nucleic acid than the probe alone, the hybridized nucleic acid will typically have more counterions surrounding it. In general, the target can be much longer than the probe, typically 2 to 100 times, in which case the counterion accumulation will be dominated by single stranded regions of the target.

Alternatively, it can be possible to increase the signal contrast between single stranded and double stranded nucleic acid by limiting the electrical signal from the probe strands. In particular, this can be done by limiting the electrical attraction between the probe strand and the counterions which participate in electron transfer. For example, if the probe strands are constructed such that they do not contain a negatively charged backbone, then they will not attract counterions. Accordingly, more of the detectable signal will be due to counterions associated with the target strands. In cases where hybridization has not occurred, the detectable signal will be measurably lower since the target strands are not present to participate in counterion attraction.

Probe strands without a negatively charged backbone can include peptide nucleic acids (PNAs), phosphotriesters, methylphosphonates. These nucleic acid analogs are known in the art.

In particular, PNAs are discussed in: Nielsen, “DNA analogues with nonphosphodiester backbones,” Annu Rev Biophys Biomol Struct, 1995;24:167-83; Nielsen et al., “An introduction to peptide nucleic acid,” Curr Issues Mol Biol, 1999;1(1-2):89-104; Ray et al., “Peptide nucleic acid (PNA): its medical and biotechnical applications and promise for the future,” FASEB J., 2000 June;14(9):1041-60; all of which are hereby expressly incorporated by reference in their entirety.

Phophotriesters are discussed in: Sung et al., “Synthesis of the human insulin gene. Part II. Further improvements in the modified phosphotriester method and the synthesis of seventeen deoxyribooligonucleotide fragments constituting human insulin chains B and mini-CDNA,” Nucleic Acids Res, 1979 Dec. 20;7(8):2199-212; van Boom et al., “Synthesis of oligonucleotides with sequences identical with or analogous to the 3′-end of 16S ribosomal RNA of Escherichia coli: preparation of m-6-2-A-C-C-U-C-C and A-C-C-U-C-m-4-2C via phosphotriester intermediates,” Nucleic Acids Res, 1977 March;4(3):747-59; Marcus-Sekura et al., “Comparative inhibition of chloramphenicol acetyltransferase gene expression by antisense oligonucleotide analogues having alkyl phosphotriester, methylphosphonate and phosphorothioate linkages,” Nucleic Acids Res, 1987 Jul. 24;15(14):5749-63; all of which are hereby expressly incorporated by reference in their entirety.

Methylphosphonates are discussed in: U.S. Pat. No. 4,469,863 (Ts'o et al.); Lin et al., “Use of EDTA derivatization to characterize interactions between oligodeoxyribonucleoside methylphophonates and nucleic acids,” Biochemistry, 1989, Feb. 7;28(3):1054-61; Vyazovkina et al., “Synthesis of specific diastereomers of a DNA methylphosphonate heptamer, d(CpCpApApApCpA), and stability of base pairing with the normal DNA octamer d(TPGPTPTPTPGPGPC),” Nucleic Acids Res, 1994 Jun. 25;22(12):2404-9; Le Bee et al., “Stereospecific Grignard-Activated Solid Phase Synthesis of DNA Methylphosphonate Dimers,” J Org Chem, 1996 Jan. 26;61(2):510-513; Vyazovkina et al., “Synthesis of specific diastereomers of a DNA methylphosphonate heptamer, d(CpCpApApApCpA), and stability of base pairing with the normal DNA octamer d(TPGPTPTPTPGPGPC),” Nucleic Acids Res, 1994 Jun. 25;22(12):2404-9; Kibler-Herzog et al., “Duplex stabilities of phosphorothioate, methylphosphonate, and RNA analogs of two DNA 14-mers,” Nucleic Acids Res, 1991 Jun. 11;19(11):2979-86; Disney et al., “Targeting a Pneumocystis carinii group I intron with methylphosphonate oligonucleotides: backbone charge is not required for binding or reactivity,” Biochemistry, 2000 Jun. 13;39(23):6991-7000; Ferguson et al., “Application of free-energy decomposition to determine the relative stability of R and S oligodeoxyribonucleotide methylphosphonates,” Antisense Res Dev, 1991 Fall; 1(3):243-54; Thiviyanathan et al., “Structure of hybrid backbone methylphosphonate DNA heteroduplexes: effect of R and S stereochemistry,” Biochemistry, 2002 Jan. 22;41(3):827-38; Reynolds et al., “Synthesis and thermodynamics of oligonucleotides containing chirally pure R(P) methylphosphonate linkages,” Nucleic Acids Res, 1996 Nov. 15;24(22):4584-91; Hardwidge et al., “Charge neutralization and DNA bending by the Escherichia coli catabolite activator protein,” Nucleic Acids Res, 2002 May 1;30(9): 1879-85; Okonogi et al., “Phosphate backbone neutralization increases duplex DNA flexibility: A model for protein binding,” PNAS U.S.A., 2002 Apr. 2;99(7):4156-60; all of which are hereby incorporated by reference.

In general, an appropriate nucleic acid analog probe will not contribute, or will contribute less substantially, to the attraction of counterions compared to a probe made of natural DNA. Meanwhile, the target strand will ordinarily feature a natural phosphate backbone having negatively charged groups which attract positive ions and make the strand detectable.

Alternatively, a probe may be constructed that contains both charged nucleic acids and uncharged nucleic acid analogs. Similarly, pure DNA probes can be used alongside probes containing uncharged analogs in an assay. However, precision in distinguishing between single stranded and double stranded will generally increase according to the electrical charge contrast between the probe and the target strands. Hence, the exclusive use of probes made entirely of an uncharged DNA analog will generally allow the greatest signal contrast between hybridized and non-hybridized molecules on the chip. In general, probe strands containing methylphosphonates are preferred when nucleic acid analogs are desired.

Ru(NH₃)₅py³⁺ is a preferred counterion, though any other suitable transition metal complexes that bind nucleic acid electrostatically and whose reduction or oxidation is electrochemically detectable in an appropriate voltage regime can be used.

Various techniques for measuring the amount of counterions can be used. In some preferred embodiments, amperometry is used to detect an electrochemical reaction at the electrode. Generally, an electrical potential will be applied to the electrode. As the counterions undergo an electrochemical reaction, for example, the reduction of a trivalent ion to divalent at the electrode surface, a measurable current is generated. The amount of current corresponds to the amount of counterions present which in turn corresponds to the amount of negatively-charged phosphate groups on nucleic acids. Accordingly, measuring the current allows a quantitation of phosphate groups and can allow the operator to distinguish hybridized nucleic acid from unhybridized nucleic acid and determine whether the target being interrogated is complementary to the probe (and contains the sequence of interest).

Although electrochemical measurement is a preferred technique for hybridization detection, those of skill in the art will appreciate that many other techniques can also be appropriate in practicing the present invention. For example, a detectable label can be attached to or otherwise associated with certain polynucleotides in the detection zone. Accordingly, such a label can then be detected as an indication of whether hybridization has occurred. Such labels are well known in the art and can include, for example, chemical moieties, dyes, radioactive probes, quantum dots, and nanoparticles. Techniques for detection of various labels can include, for example, chemical detection, radioactivity detection, UV and/or visible spectroscopy, fluorescence, and the like.

### EXAMPLES

### Example 1

### Screening a Patient for Colorectal Cancer

A patient at risk for colorectal cancer can be screened for cancer or precancer as follows:

1. An oligonucleotide sequence indicative of colorectal cancer is identified; this sequence is approximately 20 base pairs in length and is known as the “sequence of interest.”

2. A probe strand having a length of approximately 20 base pairs is produced having a sequence that is complementary to the sequence of interest.

3. A patient suspected of having colorectal cancer or suspected of later developing colorectal cancer is identified.

4. A stool sample voided from the patient is collected and sectioned to extract cells and cellular debris containing nucleic acids from the epithelial cells of the patient's colorectal tract.

5. DNA is extracted and isolated from the cells and cellular debris using methods disclosed in U.S. Pat. No. 5,741,650 (Lapidus et al.) and U.S. Pat. No. 6,406,857 (Shuber et al.).

6. DNA molecules isolated from the stool sample are amplified using digital PCR.

7. Single stranded PCR products are isolated using a streptavidin-coated bead.

8. The isolated, single stranded nucleic acid is cyclized and subject to RCA to produce elongated target strands.

9. A plurality of probe strands containing a biotin complex are immobilized on a gold electrode coated with streptavidin. A liquid medium is placed in contact with the electrode surface and with the immobilized probe strands.

10. A plurality of target strands are introduced to the liquid medium such that they are allowed to interact with the probe strands. Hybridization stringency is controlled by adjusting temperature, pH, and the quantity of TMAC in the liquid medium. Hybridization stringency is set such that perfectly complementary sequences hybridize but that all others do not.

11. On-chip amplification is conducted using head-to-tail polymerization to increase the length of any hybridized target on the electrode. This amplification adds approximately 10,000 base pairs to each bound target strand.

12. Ru(NH₁₃)₅py³⁺ ions in a liquid are added to the liquid medium as counterions able to form an electrostatic cloud around nucleic acids.

13. An electrical potential is applied to the electrode on which the nucleic acid probes are immobilized.

14. As Ru(NH₃)₅py³⁺ ions are reduced from trivalent to divalent at the electrode surface, current is measured at the electrode. The amount of current is recorded and used to determine whether the immobilized probes have hybridized to target strands or remain unhybridized.

15. The hybridization status of the probes to the targets is used to evaluate the health of the patient with regard to colorectal cancer and to decide whether to administer further healthcare services to the patient, including, for example, counseling, additional testing, administration of pharmaceutical agents, surgery, etc.

What is claimed is:

1. A method for detecting a target polynucleotide, comprising the steps of:
synthetically producing an enlarged target polynucleotide;
hybridizing the target polynucleotide to a probe polynucleotide in a detection zone; and
detecting the amount of polynucleotide in the detection zone to ascertain whether target polynucleotide has hybridized in said detection zone.

2. The method of claim 1, wherein the hybridizing step is performed prior to the step of producing an enlarged target polynucleotide.

3. The method of claim 1, wherein the hybridizing step is performed after the step of producing an enlarged target polynucleotide.

4. The method of claim 1 wherein the enlarged target polynucleotide is produced prior to the hybridizing step and the enlarged target polynucleotide is further enlarged after the hybridizing step.

5. The method of claim 4 wherein the enlarged target polynucleotide is produced prior to the hybridizing step by rolling circle amplification.

6. The method of claim 4 wherein the enlarged target polynucleotide is further enlarged after the hybridizing step by rolling circle amplification.

7. The method of claim 1, wherein target polynucleotide is produced by an amplification step and wherein the amplification step is dependent on the presence of analyte polynucleotide in a sample.

8. The method of claim 7, wherein the analyte polynucleotide is genomic DNA.

9. The method of claim 7, wherein the amplification step comprises rolling circle amplification.

10. The method of claim 1, wherein target polynucleotide is enlarged by attaching one or more polynucleotide strands thereto.

11. The method of claim 1, wherein the target polynucleotide is enlarged by attachment of a plurality of polynucleotide strands thereto, producing a branched structure.

12. The method of claim 1, wherein the target polynucleotide is enlarged by ligation of polynucleotide thereto.

13. The method of claim 12, wherein the ligation comprises addition of multiple polynucleotides in a head-to-tail ligation reaction.

14. The method of claim 1 further comprising the step of hybridizing said target polynucleotide to more than one probe polynucleotide in the detection zone.

15. The method of claim 1, wherein the detecting step comprises associating a label with all the polynucleotide in the detection zone, and then detecting the label.

16. The method of claim 15, wherein the label is detected quantitatively.

17. The method of claim 15, wherein the label is detected photometrically.

18. The method of claim 1, wherein the detecting step comprises associating a charged species with charged phosphate groups on the polynucleotide, and then detecting the presence of the charged species.

19. The method of claim 18 wherein the probe polynucleotide does not contain charged phosphate groups and the charged species associates only with the target polynucleotide.

20. The method of claim 18, wherein the probe polynucleotide is attached directly or indirectly to an electrode, and the presence of the charged species is detected through said electrode.

21. The method of claim 20, wherein the charged species is a redox moiety.

22. The method of claim 20, wherein the charged species is detected electrochemically.

23. The method of claim 21, wherein the charged species comprises a ruthenium compound.

24. The method of claim 23, wherein the ruthenium compound is ruthenium pentamine pyridine 3+.

25. The method of claim 1, comprising practicing the steps of claim 1 to effect the detection of target polynucleotides in multiple detection zones, wherein the identity of the probe polynucleotide varies from detection zone to detection zone.

26. The method of claim 25, wherein the probe polynucleotides in different detection zones are complementary to different regions of the same target polynucleotide.

27. The method of claim 26, wherein duplex polynucleotide comprising probe and target with a single base mismatch has a melting temperature Tₘ₁ and duplex polynucleotide comprising probe and target with no base mismatch has a higher melting temperature Tₘ₂, further comprising the steps of:
performing one detection step at a temperature below Tₘ₁, and
performing another detection step at a temperature between Tₘ₁ and Tₘ₂.

28. A method for detecting a nucleic acid analyte, comprising:
generating an elongated reporter nucleic acid if the nucleic acid analyte is present;
capturing the reporter nucleic acid with an immobilized probe that is substantially shorter than the reporter nucleic acid; and
generating a signal that is a function of the size of the captured reporter nucleic acid to indicate the presence or absence of the nucleic acid analyte.

29. The method of claim 28, wherein the reporter nucleic acid includes a target sequence not present in the nucleic acid analyte.

30. The method of claim 29, wherein the probe is nucleic acid or a nucleic acid analog and the target sequence is complementary to and hybridizes with probe sequence.

31. The method of claim 30, wherein the reporter nucleic acid is at least twice as large as the probe.

32. The method of claim 30, wherein the reporter nucleic acid is at least 4 times as large as the probe.

33. The method of claim 28, wherein the reporter nucleic acid is generated using rolling circle amplification.

34. The method of claim 28, wherein the signal is an electrochemically-generated signal.

35. The method of claim 34, wherein the signal is an amperometric signal.

36. The method of claim 34, wherein the signal is a coulometric signal.

37. The method of claim 34, wherein the signal is generated by a charged redox moiety that is electrostatically attracted to phosphate groups of the reporter nucleic acid.

38. The method of claim 34, wherein the probe is immobilized to an electrode.
